(19) United States
(12) Patent Application Publication
(10) Pub. No.: US 2021/0388164 A1
(43) Pub. Date: Dec. 16, 2021

(54) ALDEHYDE CROSSLINKING, PROTEIN BASED TISSUE SCAFFOLDS, AND USES THEREOF

(71) Applicant: University of Florida Research Foundation, Inc., Gainesville, FL (US)

(72) Inventors: Josephine ALLEN, Gainesville, FL (US); Vidhya RAMASWAMY, Plymouth, MN (US); Krista DULANY, Boca Raton, FL (US); Allison N. GOINS, Marietta, GA (US)

(21) Appl. No.: 17/281,714

(22) PCT Filed: Oct. 1, 2019
(86) PCT No.: PCT/US19/54010

§ 371 Date: Mar. 31, 2021

Related U.S. Application Data
(60) Provisional application No. 62/739,716, filed on Oct. 1, 2018.

(51) Int. Cl. C08J 3/24; A61L 27/22; A61L 27/24; A61L 27/54; A61L 27/56

(52) U.S. Cl. CPC C08J 3/24; A61L 27/222; A61L 27/227; C08J 2389/04; A61L 27/54; A61L 27/56; A61L 2400/12; A61L 27/24

(57) ABSTRACT

Described herein are methods of preparing protein scaffolds that can include the step of crosslinking protein fibers in the vapor phase of a natural aldehyde, such as cinnamaldehyde or vanillin or a solution thereof. Also described herein are protein scaffolds that can be prepared by a method that can include the step of crosslinking protein fibers in the vapor phase of a natural aldehyde solution, such as cinnamaldehyde or vanillin or a solution thereof.

### CROSS-REFERENCE TO RELATED APPLICATION

This application claims priority to U.S. Provisional Application entitled “ALDEHYDE CROSSLINKING, PROTEIN BASED TISSUE SCAFFOLDS, AND USES THEREOF,” having Ser. No. 62/739,716, filed on Oct. 1, 2018, which is entirely incorporated herein by reference.

### STATEMENT REGARDING FEDERALLY SPONSORED RESEARCH

This invention was made with government support under grant number W81XWH-15-1-0066 awarded by the U.S. Army Medical Research and Materiel Command. The government has certain rights in the invention.

### BACKGROUND

In the medical device industry protein-based implants are widely used for a variety of applications (heart valves, wound healing patches, etc.). However, some methods of stabilizing protein or protein containing scaffolds result in scaffolds that are unsuitable for long-term implantation applications due to their immunogenicity with can cause implant calcification and cell death. As such, there exists a need for improved methods of stabilizing protein-based scaffolds for biomedical implants.

### SUMMARY

Described herein are methods of making tissue scaffolds, aldehyde crosslinking, and uses thereof. In embodiments according to the present disclosure, methods of crosslinking protein fibers are described. In an embodiment, a method of crosslinking protein fibers comprises exposing a plurality of protein fibers to a vapor phase of a crosslinking solution comprising an amount of a natural aldehyde for an amount of time. In embodiments, the amount of the natural aldehyde can range from about 10% to 25% (v/v) or (w/v). In embodiments, the natural aldehyde can be cinnamaldehyde or vanillin. In embodiments, the crosslinking solution further comprises ethanol. In embodiments, the amount of ethanol ranges from about 75% to about 90% (v/v) or (w/v). In embodiments, the amount of time can range from about 1 hour to 24 hours. In embodiments, the amount of time can range from about 1 day to about 14 days. In embodiments, the protein fibers can be collagen or elastin, individually or in combination. In an embodiment, the protein fibers are gelatin. In embodiments, the protein fibers can comprise gelatin, elastin, collagen, silk, keratin, soy, or zein, individually or in combination. In embodiments, the exposing can be at a temperature of about 25° C. to about 45° C. In an embodiment, the exposing can be at a temperature of about 25° C. In an embodiment, the exposing can be at a temperature of about 37° C. In embodiments, the exposing can be at a temperature of about 25° C. to about 37° C. In embodiments, the exposing can be at a pressure of about 7.5 PSI to about 30 PSI. In embodiments, the protein fibers can be prepared by electrospinning, wet/dry jet spinning, dry spinning, centrifugal spinning, solution blowing, self-assembly, phase separation, or 3D printing before exposing.

Described herein are protein scaffolds. In an embodiment, a protein scaffold comprises protein fibers crosslinked to each other via a natural aldehyde. In an embodiment, the natural aldehyde cam ne cinnamaldehyde or vanillin. In an embodiment, the protein fibers can be collagen or elastin, individually or in combination.

Protein scaffolds as described herein can further comprise a population of cells. The cells can be mammalian cells. The population of cells can be a homologous or heterologous population of cells of mesodermal derivation. In an embodiment, the cells can be cardiomyocytes. In an embodiment, the cells can be smooth muscle cells. In an embodiment, the cells can be vascular endothelial cells. In an embodiment, the cells can be cardiomyocytes, smooth muscle cells, or vascular endothelial cells, individually or in combination.

Protein scaffolds as described herein can further comprise an active agent.

Described herein are protein scaffolds produced by any one of the methods described herein.

Also described herein are methods of implanting a protein scaffold as described herein into a subject. In an embodiment, the subject has cardiovascular disease.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a graph demonstrating the results of spectrochemical analysis of electrospun scaffolds crosslinked at various crosslinking conditions performed using attenuated total reflectance Fourier transformation infrared spectroscopy (ATR-FTIR).

FIGS. 2A-2C show SEM images of (FIG. 2A) uncrosslinked gelatin fibers, (FIG. 2B) liquid trans-cinnamaldehyde (tCa) crosslinked fibers after about 24 h at room temperature, and (2C) tCA vapor crosslinked fibers after 7 days at 45° C. All images are at a magnification of 5000×.

FIGS. 3A-3F show SEM images uncrosslinked and crosslinked gelatin nanofibers. Crosslinking was performed with vanillin, cinnamaldehyde, or glutaraldehyde.

FIGS. 4A-4C show graphs demonstrating the results from a trinitrobenzene assay to detect free amine groups that have been correlated to the % crosslinking

FIGS. 5A-5D show graphs demonstrating results of FTIR spectrochemical of electrospun scaffolds crosslinked at various conditions.

FIGS. 6A-6B shows a representative graph demonstrating the results of thermogravimetric analysis of gelatin samples submerged for 1 day in (a) 25% tCa at 37° C. and (b) 25% tCa at 25° C.

FIG. 7 shows a representative graph demonstrating the results of thermogravimetric analysis of gelatin samples submerged for 1 day in 10% tCa at 37° C.

FIGS. 8A-8B shows a summary graph (FIG. 8A) and table (FIG. 8B) of the thermogravimetric analysis data shown in FIGS. 6A-7.

FIGS. 9A-9B shows representative graphs demonstrating the results of thermogravimetric analysis of gelatin samples crosslinked using vapor for 5 days in (a) 25% tCa at 37° C. and (b) 25% tCa at 25° C.

FIG. 10 is a flowchart of an embodiment of a method 100 as described herein.

FIG. 11 is a flowchart of an embodiment of a method 200 as described herein.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, organic chemistry, biochemistry, physiology, cell biology, materials science, and the like, which are within the skill of the art. Such techniques are explained fully in the literature.

### Definitions

As used herein, “about,” “approximately,” and the like, when used in connection with a numerical variable, can generally refers to the value of the variable and to all values of the variable that are within the experimental error (e.g., within the 95% confidence interval for the mean) or within +1-10% of the indicated value, whichever is greater.

As used herein, “active agent” or “active ingredient” can refer to a substance, compound, or molecule, which is chemically or biologically active or otherwise, induces a chemical, biological or physiological effect on a subject to which it is administered to. In other words, “active agent” or “active ingredient” refers to a component or components of a composition to which the whole or part of the effect of the composition is attributed.

The term “biocompatible”, as used herein, refers to a material that along with any metabolites or degradation products thereof that are generally non-toxic to the recipient and do not cause any significant adverse effects to the recipient. Generally speaking, biocompatible materials are materials that do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

As used herein, “polypeptides” or “proteins” can refer to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, VV), Tyrosine (Tyr, Y), and Valine (Val, V). “Protein” and “Polypeptide” can refer to a molecule composed of one or more chains of amino acids in a specific order. The term protein is used interchangeably with “polypeptide.” The order is determined by the base sequence of nucleotides in the gene coding for the protein. Proteins can be required for the structure, function, and regulation of the body's cells, tissues, and organs.

As used herein, “crosslinking” means the formation of chemical bonds between protein fibers, in particular covalent chemical bonds, with or without the use of a linker between fibers.

As used herein, “aldehyde” is a compound containing a functional group with the structure —CHO, comprising a carbonyl center (a carbon double-bonded to oxygen) with the carbon atom also bonded to hydrogen and to an R group, which can be any generic alkyl, aryl, or other side chain, substituted or unsubstituted, saturated or unsaturated.

As used herein, “alkyl” or “alkyl group” refers to a saturated aliphatic hydrocarbon which can be straight or branched, having 1 to 40, 1 to 20, 1 to 10, or 1 to 5 carbon atoms, where the stated range of carbon atoms includes each intervening integer individually, as well as sub-ranges. Examples of alkanes include, but are not limited to methyl, ethyl, n-propyl, i-propyl, n-butyl, s-butyl, tbutyl, n-pentyl, and s-pentyl. Reference to “alkyl” or “alkyl group” includes unsubstituted and substituted forms of the hydrocarbon group.

As used herein, “aryl” or “aryl group” refers to an aromatic monocyclic or multicyclic ring system of 6 to 20 or 6 to 10 carbon atoms. The aryl is optionally substituted with one or more C1-C20 alkyl, alkylene, alkoxy, or haloalkyl groups. Exemplary aryl groups include phenyl or naphthyl, or substituted phenyl or substituted naphthyl. Reference to “aryl” or “aryl group” includes unsubstituted and substituted forms of the hydrocarbon group.

As used herein, “aromatic” refers to a monocyclic or multicyclic ring system of 6 to 20 or 6 to 10 carbon atoms having alternating double and single bonds between carbon atoms. Exemplary aromatic groups include benzene, naphthalene, and the like. Reference to “aromatic” includes unsubstituted and substituted forms of the hydrocarbon

The term “substituted,” as in “substituted alkyl”, “substituted aryl,” “substituted heteroaryl” and the like, means that the substituted group may contain in place of one or more hydrogens a group such as alkyl, hydroxy, amino, halo, trifluoromethyl, cyano, alkoxy, alkylthio, or carboxy.

As used herein, “aliphatic” or “aliphatic group” refers to a saturated or unsaturated, linear or branched, cyclic (non-aromatic) or heterocyclic (nonaromatic), hydrocarbon or hydrocarbon group and encompasses alkyl, alkenyl, and alkynyl groups, and alkanes, alkene, and alkynes, for example.

### Discussion

Protein-based scaffolds are of interest because they provide biological recognition and spatiotemporal cues in implant applications, however they must be crosslinked to stabilize the proteins such that they can be used for various applications. Chemical crosslinkers, plasticizers, and ionizing radiations have been employed to stabilize protein based scaffolds. Currently, the most robust and popular crosslinking agents are formaldehyde and glutaraldehyde. These can cause significant cytotoxicity and tissue calcification. As such, there exists a need for alternative crosslinking agents for to stabilize protein-based implants.

With that said, described herein are methods of crosslinking protein-based scaffolds using natural aldehydes in the vapor phase. In some aspects, the natural aldehydes can be cinnamaldehyde, transcinnamaldehyde, or vanillin. Also described herein are protein scaffolds that can be produced by the methods and compositions described herein. The methods of crosslinking herein can be less toxic than current methods. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Trans-cinnamaldehyde (tCa) (compound (1)) and vanillin (Va) (compound (2)) are natural aromatic aldehydes that contain one aldehyde group as compared to glutaraldehyde (compound 3). Unlike glutaraldehyde, the free aldehydes VA and tCA release and can be metabolized. In vivo, cinnamaldehyde and vanillin are oxidized and can form residual alcohols and acids.

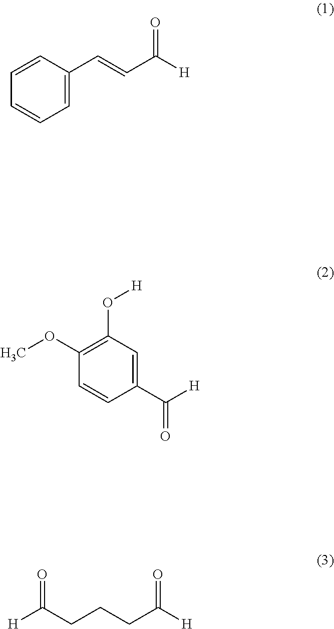

Described herein are methods of crosslinking proteins using cinnamaldehyde or vanillin in the vapor phase to form protein scaffolds that can be useful in tissue engineering. In some aspects, the method can include preparing a crosslinking solution comprising vanillin or cinnamaldehyde with a volatile solvent. In some aspects, the crosslinking solution is 100% cinnamaldehyde v/v. In some aspects, the crosslinking solution can be prepared by diluting a 100% (v/v) solution of cinnamaldehyde in ethanol to concentrations of about 25% (v/v) in ethanol. In some aspects, the crosslinking solution can be prepared from powdered vanillin at concentrations of 25% (w/v) in ethanol. Additional concentrations can be possible and are only limited by the solubility of the natural aldehyde in the volatile solvent.

This technology is demonstrated with protein nanofibers but may be used with other porous scaffolds composed of materials with free amine groups to facilitate crosslinking. Protein nanofibers can be fabricated and prepared for crosslinking using any suitable method, including but not limited to electrospinning, wet/dry jet spinning, dry spinning, centrifugal spinning, solution blowing, self-assembly, phase separation, and 3D printing. In some aspects, the solution used to prepare the protein nanofibers can include from about 5 wt % to 15 wt % protein in a solvent, such as a suitable solution for electrospinning. In some aspects, the solution used to prepare the protein nanofibers can include about 5, 6, 7, 8, 9, 10, 11, 12, 12.5, 13, 14, 15 wt % of the protein. Suitable solvents include, but are not limited to what is presented here, tetrafluoroethylene (TFE) and 1,1,1,3,3,3-Hexafluoroisopropanol (HFIP). Protein nanofibers can be made of any suitable proteins, including but not limited to gelatin, elastin, collagen, silk, keratin, soy, zein, and combinations thereof.

The protein nanofibers can then be crosslinked in the vapor phase of a crosslinking solution previously described. The crosslinking reaction can be allowed to occur for a period of time ranging from about 1, 2, 3, 4, 5, 6, 7, 8, 9, 10, 11, 12, 13, 14, 15, 16, 18, 19, 20, 21, 22, 23, 24 hours or more. The crosslinking reaction can be allowed to occur for a period of time ranging from about 1, 2, 3, 4, 5, 6, 7, 8, 9, 10, 11, 12, 13, 14 days or more. Crosslinking can occur at a temperature of about 25° C. to about 45° C., about 30° C. to about 40° C., about 35° C., about 25° C., or about 37° C. The pressure at which the crosslinking reaction can occur can range from about 7.5 PSI to about 30 PSI, about 10 PSI to about 27.5 PSI, about 12.5 PSI to about 25 PSI, about 15 PSI to about 22.5 PSI, about 17.5 PSI to about 20 PSI.

Stability of the resulting crosslinked protein scaffold can be determined by evaluating its decomposition in water. Confirmation of crosslinking can be determined indirectly using a TNBSA assay to evaluate the number of free amino groups present in the crosslinked samples. A minimum % crosslinking of approximately 60% was necessary to achieve a scaffold that was stable in an aqueous environment. Scaffolds with less than 60% crosslinking dissolved in water and would not be suitable for biological applications. It is important to note that the necessary crosslinking percentage will vary and will be material or protein specific. In order to successfully crosslinking a protein scaffold it is necessary to establish crosslinking conditions that produce a scaffold that is thermally stable at about 37° C. (does not melt) and aqueous stability (does not dissolve in a water or other aqueous solution).

Also described herein are crosslinked protein scaffolds that can be prepared by crosslinking the protein nanofibers in the vapor phase of a crosslinking solution described herein according to a method described herein. The scaffolds can be used in various tissue engineering applications. In some aspects, the protein scaffolds can be used as a vascular tissue scaffold. In some aspects, the protein scaffolds can be seeded with one or more types of cells. In some aspects, the cells can be cardiomyocytes, vascular endothelial cells, and/or smooth muscle cells, individually or in combination. The cells can be a plurality of cells. In some aspects, the protein scaffolds can be seeded with one or more suitable active agents. Suitable active agents can include but are not limited to, small molecule pharmaceutical agents, biological agents (e.g. nucleic acids, polypeptides, hormones, etc.), growth factors (for example vascular endothelial growth factors, VEGF), imaging agents, and combinations thereof.

The protein scaffolds described herein can be used as a scaffold for tissue engineering. The protein scaffolds described herein can be implanted into a subject or subject in need thereof. In some aspects, the subject or subject in need thereof has cardiovascular disease. In further aspects, the subject or subject in need thereof has a wound in need of healing.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

Protein Cross/inking Using Cinnamaldehyde Vapors.

This Example evaluates the use of trans-cinnamaldehyde (tCA) as a crosslinker to stabilize protein scaffolds. Tissue engineering scaffolds made of proteins from animal and plant sources have been used effectively for a variety of applications. However, these scaffolds must be crosslinked to stabilize the proteins before they can be used in biological applications. Protein scaffolds, without proper crosslinking, can have inferior resistance to degradation and mechanical strength. Therefore, to improve their physical properties, many crosslinking strategies have been utilized such as chemical crosslinkers, plasticizers as well as ionizing radiations. A challenge with the various crosslinking strategies is the downstream toxic effects from the chemicals and methods used in the crosslinking process. Currently, the most robust and popular crosslinking agents employed are primarily aldehydes such as formaldehyde and glutaraldehyde which can cause significant toxicity to cells unless appropriate precautions are taken. Therefore, there is a constant search for alternative natural crosslinking agents which do not cause significant toxicity to tissues.

Cinnamaldehyde occurs naturally as an aromatic α,β-unsaturated aldehyde. It is derived from cinnamon which is used popularly as a spice. It also has medicinal properties and has been used as a home remedy for ailments for the common cold and digestive disorders. Cinnamon oil has also been used as a food preservative because of its excellent anti-microbial properties. More recently it has been identified as a crosslinking agent for protein films in the food packaging industry. Another recent report also explores its use to crosslink protein hydrogels for wound healing applications^{1,2}.

This Example investigates the use of cinnamaldehyde vapors for crosslinking proteins. In this work, the crosslinking action of cinnamaldehyde liquid is compared with that of cinnamaldehyde vapors. The use of the vapor form of cinnamaldehyde for crosslinking applications has not been reported before.

Electrospinning Gelatin Nanofibers. 12.5 wt % solution of gelatin in tetrafluoroethylene was prepared for electrospinning. The mixture was vortexed for 15 min and allowed to dissolve overnight to thoroughly mix the constituents. The gelatin solution was fed through a 25G needle at 0.8 mL/hr with a syringe pump with a voltage potential between 22-25 kV and working distance of 25 cm.

Cinnamaldehyde Crosslinking and Preliminary Stabilization Assessment. The gelatin scaffolds in their as-spun state, before crosslinking, were soluble in water. The scaffolds were then crosslinked using the conditions mentioned in Table able 1 below. The stability of the scaffolds was preliminarily tested by assessing their ability to resist dissolution in water.

TABLE 1
tCA cross-linking conditions and resistance to degradation.

| Conditions | Water stability |
|---|---|
| Un-crosslinked control | Dissolved in water |
| Liquid tCA crosslinking for 24 h at RT | Stable in water |
| Vapor tCA crosslinking for 4 days at 45° C. | Dissolved in water |
| Vapor tCA crosslinking for 7 days at 45° C. | Stable in water |
| Thermal crosslinking control for 7 days at 45° C. | Dissolved in water |

From preliminary testing, it was shown that only two conditions stabilized the gelatin fibers, 1) the fibers crosslinked with liquid tCA overnight at RT; and 2) the tCA vapor crosslinked fibers for 1 week at 45° C.

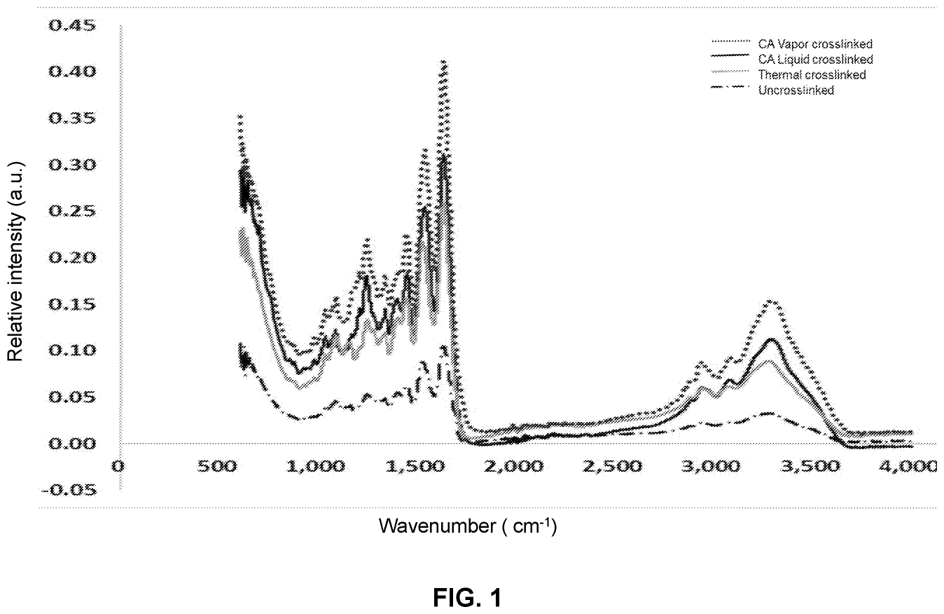

FITR Characterization. The crosslinking of scaffolds was confirmed by FTIR. Spectrochemical analysis of the electrospun scaffolds crosslinked at various conditions was performed using attenuated total reflectance Fourier transformation infrared spectroscopy (ATR-FTIR). ATR-FTIR spectra were obtained for each of the samples using the Nicolet 6700 FTIR Spectrometer (Thermo Scientific) and a diamond tip window. The spectra were read over a range of 600-4000 cm⁻¹ for each of the spectra and a total of 32 scans were used with a resolution of 4 cm⁻¹. Results are shown in FIG. 1. FTIR analysis confirmed the crosslinking of scaffolds as indicated by a characteristic Amide II (NH) bending vibrations peak observed around 1550 cm⁻¹. Additionally, Amide I (C═O stretching) and Amide A (N—H stretching) signatures were also seen respectively at 1632-1664 and 3320-3340 cm⁻¹ for all scaffolds tested.

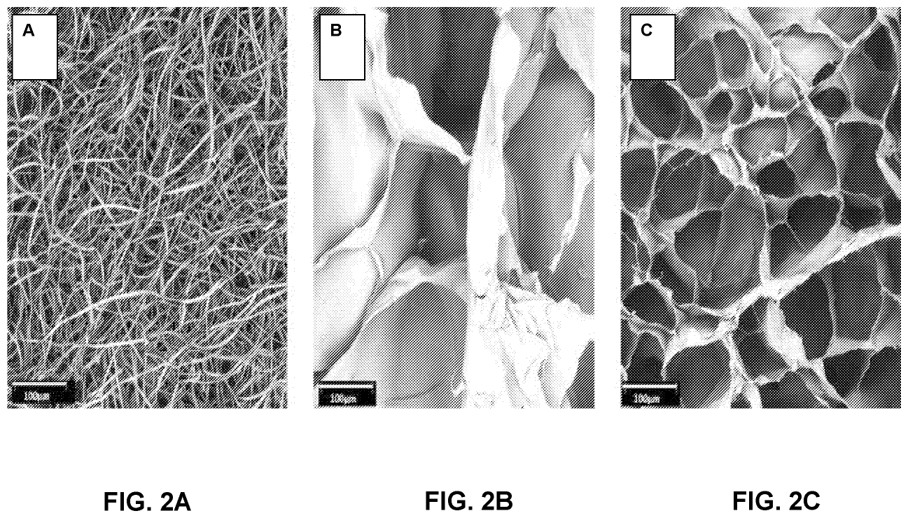

SEM Characterization. The stable samples were washed and frozen at −80° C. for 18 h before being lyophilized. The dried scaffolds were then mounted for SEM analysis and the structure of the scaffolds was observed using a tabletop SEM. The SEM images (FIGS. 2A-2C) show that there is coalescing of fibers and the morphology changes on crosslinking. Further comprehensive evaluation of the crosslinked fibers, as well as their structure is required.

### References for Example 1

1. Nipun Babu V, Kannan S. Enhanced delivery of baicalein using cinnamaldehyde cross-linked chitosan nanoparticle inducing apoptosis. Int J Biol Macromol. 2012; 51(5):1103-1108. doi:10.1016/j.ijbiomac.2012.08.038.
2. Cheirmadurai K, Thanikaivelan P, Murali R. Highly biocompatible collagen-Delonix regia seed polysaccharide hybrid scaffolds for antimicrobial wound dressing. Carbohydr Polym. 2016; 137:584-593. doi:10.1016/j.carbpol.2015.11.015.

### Example 2

Investigation of Natural Aldehydes as Crosslinking Agents

Glutaraldehyde is among the most popular methods of chemical crosslinking using a chemical agent.[1] It is widely accepted that glutaraldehyde stabilizes gelatin and other proteins through Schiff base reactions in which unprotonated ε-amino groups in the lysine and hydrolysine residues, as well as the amino groups of the N-terminal amino acids, react with the aldehyde groups present.[2] While this reaction is highly efficient, glutaraldehyde is highly toxic in biological applications. In an attempt to reduce the toxicity of glutaraldehyde, the vapor phase of glutaraldehyde has been utilized to crosslinking samples. This approach decreases the toxicity of glutaraldehyde crosslinked structures, however, in biodegradable materials, as the scaffold degrades residual free aldehydes are released and they can cause toxicity. There is a critical need for crosslinking methods that not only stabilize biopolymers and increase their aqueous, thermal, and mechanical integrity but also are non-toxic and stable over time.

Trans-cinnamaldehyde (tCa) (Compound (1)) and vanillin (Va) (Compound (2)) are two naturally occurring aromatic aldehydes that contain one aldehyde group compared to the two aldehydes present in glutaraldehyde (Compound (3)). Both tCa and Va have been primarily used in the food industry in the liquid phase to stabilize proteins and for their antibacterial properties. [3-10] They are used extensively in the food industry due to their low toxicity.[3] Both molecules crosslink materials with a similar mechanism as glutaraldehyde, Schiff base reaction.[8] These molecules have been used exclusively in their liquid phase to crosslink polymers.[5-8,11]. Unlike glutaraldehyde, the free aldehydes VA and tCA release are metabolized. In vivo, cinnamaldehyde and vanillin are oxidized and form residual alcohols and acids.[11-12]

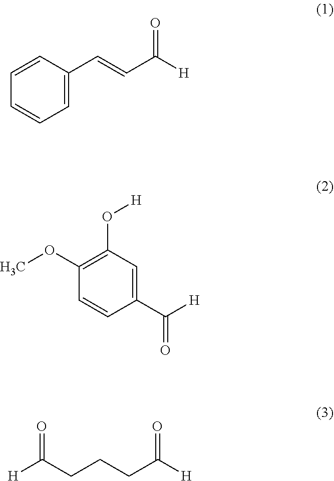

An objective of this Example was to prepare electrospun gelatin mats as a model material system and crosslink them with the vapor phase of tCA and Va. The thermal and aqueous stability of the crosslinked gelatin mats was evaluated. In this study, we investigated the efficacy of tCa and Va in the vapor phase as crosslinking agents for gelatin compared to glutaraldehyde.

Electrospinning. A solution of 10 wt. % gelatin was dissolved into 1,1,1,3,3,3-Hexafluoroisopropanol (HFIP) then loaded into a 10 mL syringe and fed through a syringe pump at 2 mL/hr. with a working distance of 15 cm and a voltage of approximately 22 kV onto a flat copper substrate. Scanning electron microscopy (SEM) images were taken to confirm a uniform bead-less nanofibrous morphology was achieved.

Scaffold Crosslinking. Aqueous solutions of tCA were prepared by diluting a 100% (v/v) solution of tCA in ethanol to concentrations of 25 (v/v) %. A vanillin solution was prepared from powered vanillin at concentrations of 25 (w/v) % in ethanol. Glutaraldehyde solutions of 25% (v/v) were used for vapor crosslinking. Electrospun gelatin scaffolds were crosslinked in vapor phase for 3, 5, 7, and 10 days of tCA and VA. These times were established through optimization (data not reported). As a positive control for crosslinking scaffolds were crosslinked in glutaraldehyde vapor for 2 and 6 hours. For all time points crosslinking was carried out a room temperature (about 25° C.) and ambient body temperature (about 37° C.).

Scanning Electron Microscopy. Fiber morphology of the electrospun gelatin scaffolds crosslinked with the varying conditions was assessed using scanning electron microscopy (SEM). Samples were cut into sections and mounted onto aluminum stubs, sputter coated with gold-palladium, then subsequently imaged.

Assessment of Crosslinking. To determine the relative number of free amino groups on the crosslinked scaffolds a 2,4,6-tri-nitrobenzene sulfonic acid (TNBSA) assay was performed. The protocol was modified from Huang et. al. [13] Briefly, the crosslinked scaffolds were incubated in a solution of 4% sodium bicarbonate solution for 30 minutes at room temperature to solubilize the unreacted amines on the scaffolds. Then an equal volume of 0.5% (w/v) TNBSA in sodium bicarbonate was added and incubated at 40° C. for two hours. The reaction was stopped by the addition of 1N hydrochloric acid. The absorbance was then read at 420 nm. To determine the relative free amino groups/mg of scaffold weight, the absorbance was divided by the weight of the scaffold.

Attenuated Total Reflectance-Fourier Transform Infrared Spectroscopy.

Crosslinking was assessed qualitatively using attenuated total reflectance Fourier transformation infrared spectroscopy (ATR-FTIR). ATR-FTIR spectra were obtained for each of the samples using the FTIR Spectrometer (Nicolet 6700, Waltman, Mass.) and a diamond tip window. The relative intensity was measured over a range of 600-4000 cm-1 for each sample and a total of 32 scans were used with a resolution of 4 cm-1.

Thermogravimetric Analysis

Thermogravimetric analysis (TGA) was completed on a subset of samples to determine the resultant change in thermal stability of gelatin samples through various crosslinking conditions. A Simultaneous Thermal Analyzer 449 F5 Jupiter was used to measure mass loss of samples as a function of temperature. Samples ranging between 1-3 g were loaded into ceramic crucibles and analyzed under a heating profile beginning around 26° C. ending at 500° C. The change mass loss was assessed for a shift in temperature where mass loss occurs. The more crosslinked samples decompose at higher temperatures due to the increase stability of the system and result in decreased mass loss in comparison to uncrosslinked samples.

Statistical Analysis

All data are reported as mean±standard deviation. To determine statistical significance between an analysis of variance (ANOVA) was performed with a Tukey post-hoc analysis to determine statistical significance between crosslinking treatment between groups.

Results

Prior to characterizing the crosslinked scaffolds, scaffolds were hydrated and washed with deionized water to assess their stability in water. Samples that remained intact in water subsequent to hydration were selected for further characterization.

Scaffold Morphology Characterization

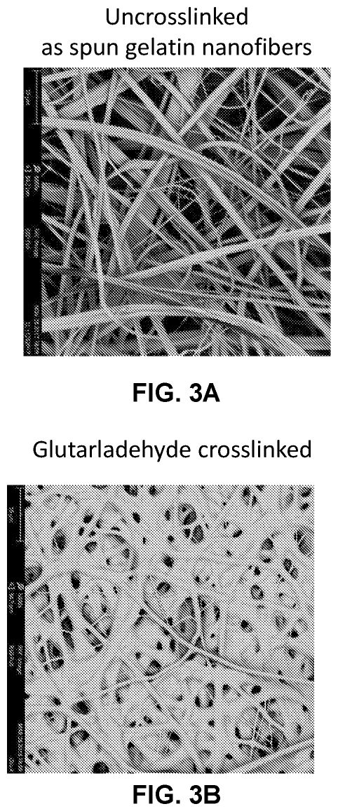

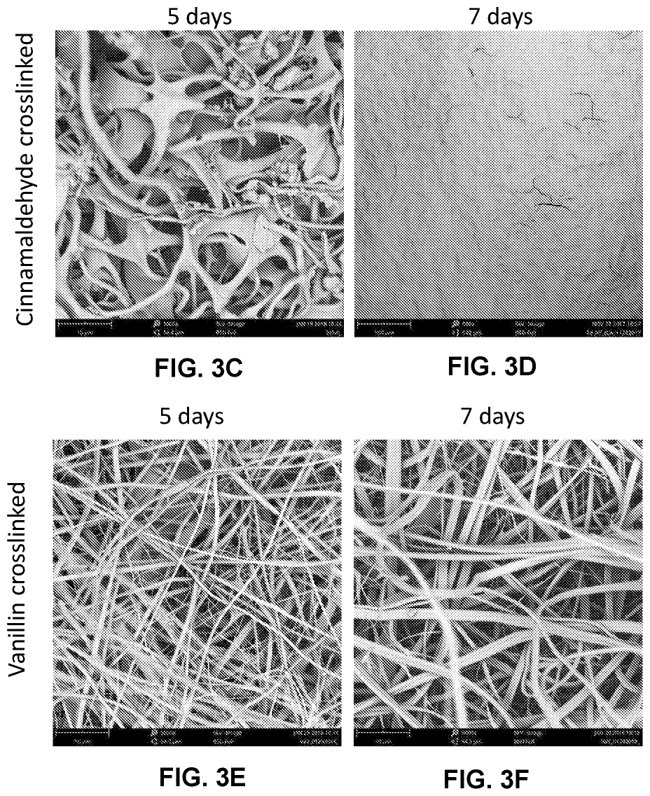

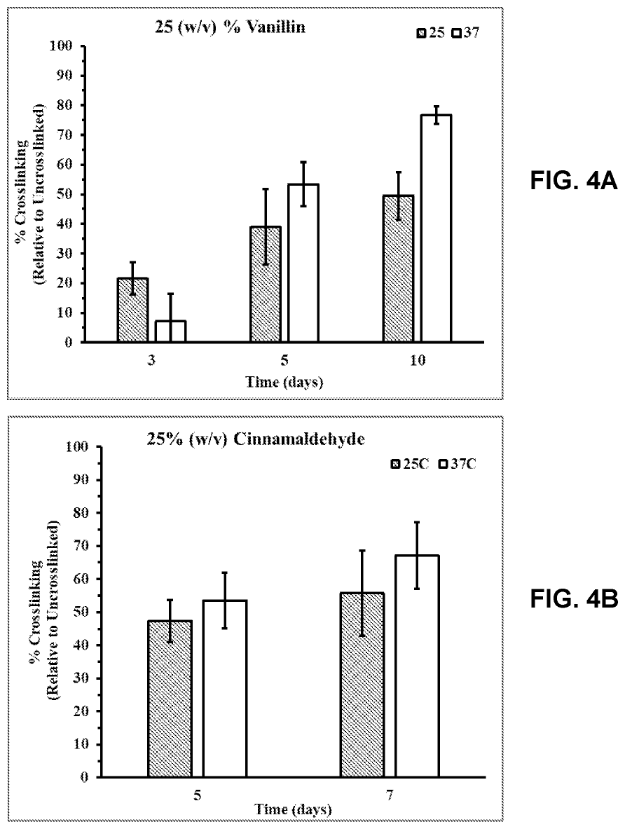

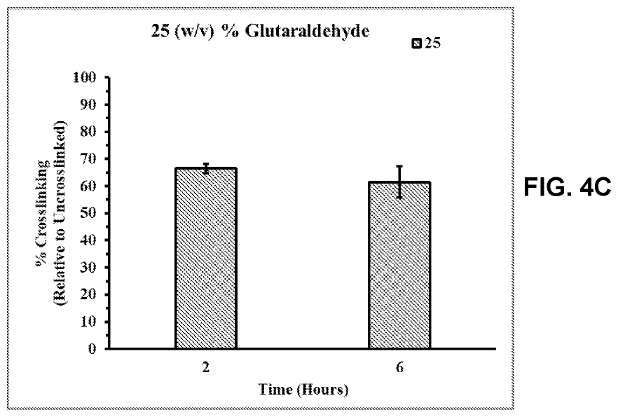

SEM images of gelatin nanofibers are shown in FIGS. 3A-3F. FIGS. 4A-4C show graphs demonstrating the results from a TNBSA assay to detect the number of free amine groups present. An uncrosslinked sample of electrospun gelatin was used to set a baseline of the maximal 100% free amines and the % crosslinking was calculated relative to the uncrosslinked samples. Equation 1 was used to calculate the percent crosslinking.

$$\% crosslinking = \frac{absorbance experimental}{absorbance uncrosslinked} \qquad [ Eq . 1 ]$$

Assessment of Crosslinking

ATR-FTIR

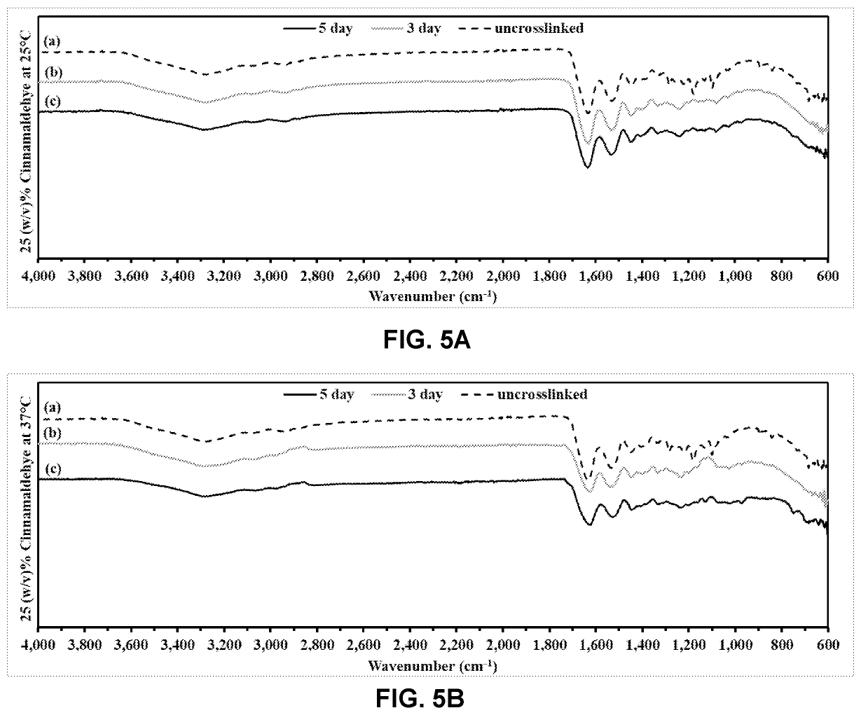

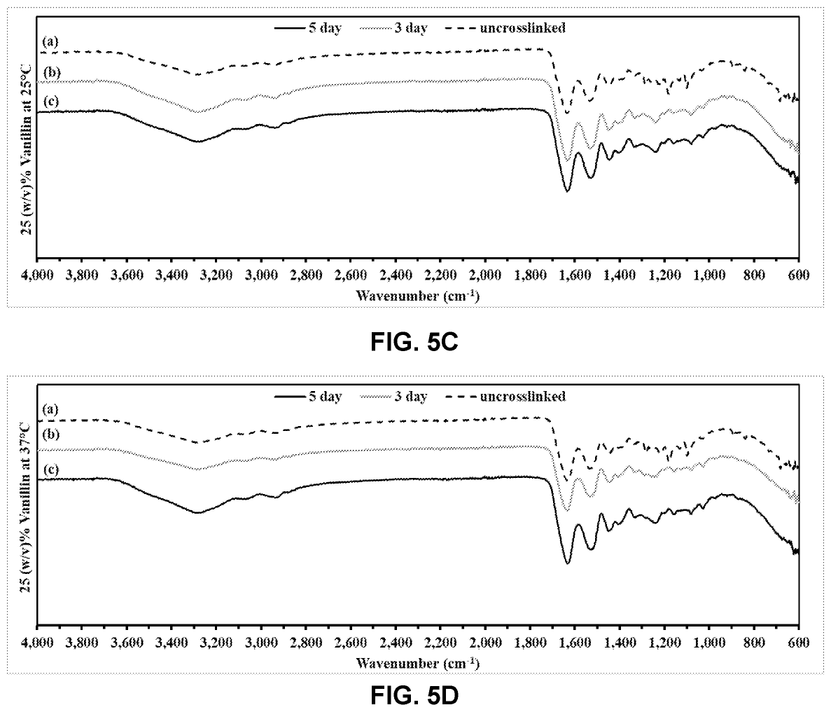

Crosslinking was assessed qualitatively and quantitatively. Qualitative assessment was performed via FTIR. FTIR spectra were obtained for uncrosslinked and crosslinked samples, FIGS. 5A-5D. The FTIR spectrum of the uncrosslinked electrospun gelatin showed a strong amide I peak from C═O stretch between 1640-1690 cm-1, and an amide II peak indicating N—H bending around 1550 cm-1. Lastly, the peaks between 1000-1300 cm-1 indicate C—N bond stretch in amines. In regards to the incorporation of the aromatic benzene from the trans-cinnamaldehyde and vanillin FTIR spectra showed an increase in C—H stretch between 3000-3100 cm-1 and C═C stretch between 1400-1600 cm-1 compared to the uncrosslinked samples. In addition to the previously mentioned peaks, a strong peak at 1450 cm-1 was observed in the crosslinked gelatin due to aldimine absorption. It was found that the amide II peak changed from smooth to several small peaks. Glutaraldehyde has an aldehyde group (—CHO) that reacts with the amino group of the lysine residues of proteins [1]. The crosslinked membranes experienced a slow change in color from white to yellow. The color change occurred because the aldimine linkage (CH═N) reactions took place during the crosslinking process.

3.3.2 Thermogravimetric analysis of Various CrossLinking Conditions.

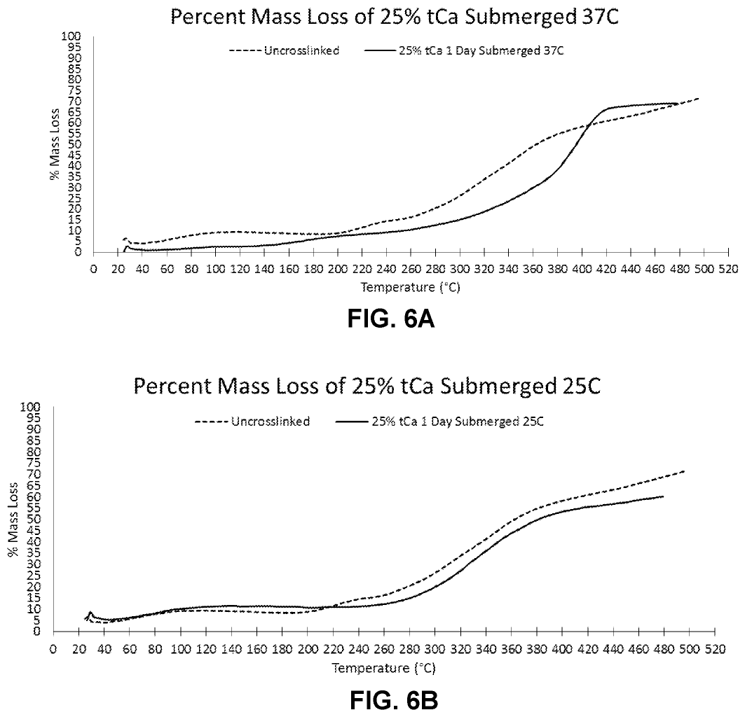

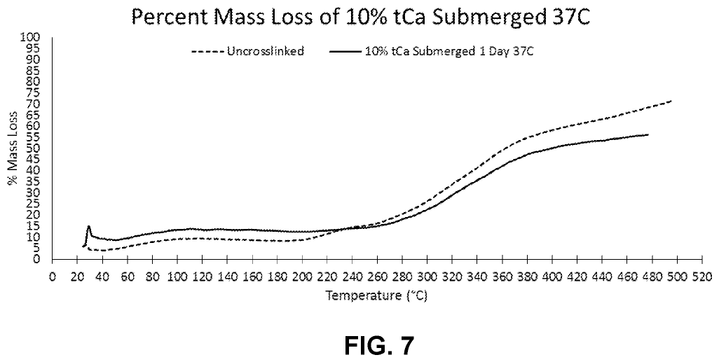

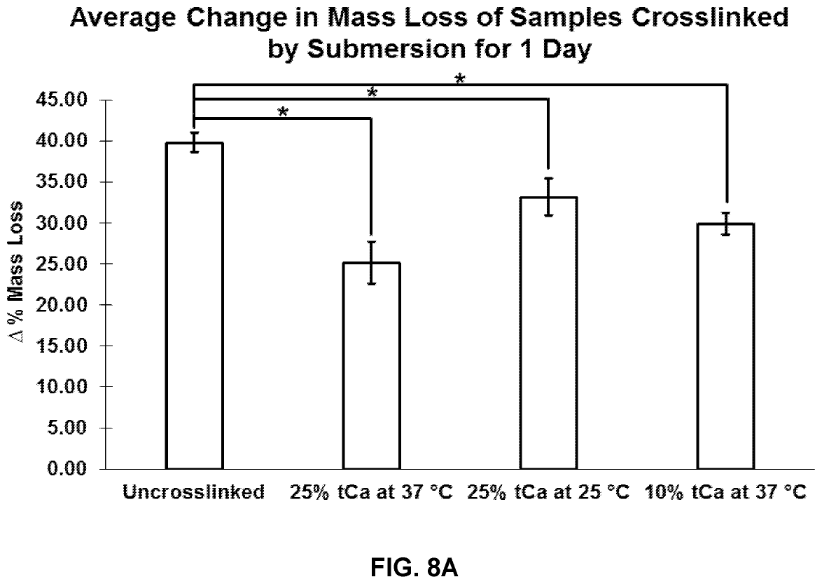

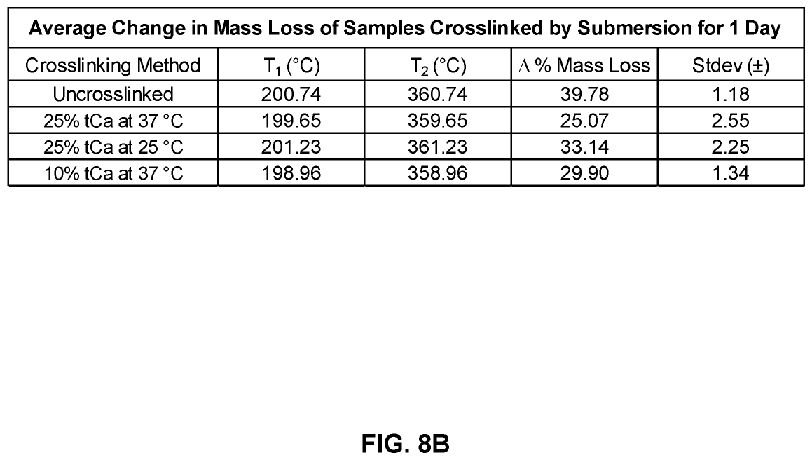

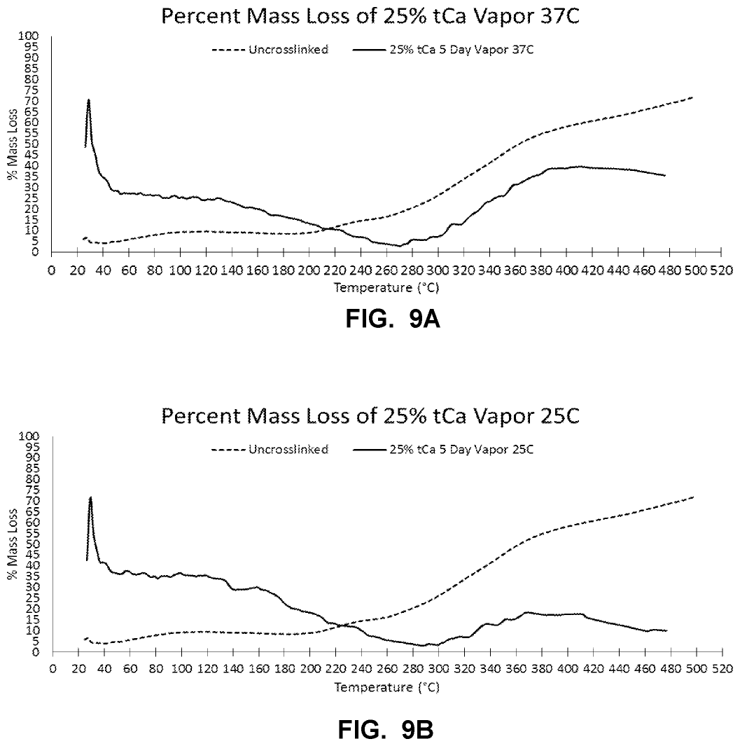

Thermal stability of the scaffolds was determined by measuring the percent mass loss of gelatin scaffolds crosslinked using various methods. FIG. 6 shows a graph that can demonstrate the results of a thermogravimetric analysis of gelatin scaffolds crosslinked for one day in a 25% (v/v) tCA at both 37° C. (FIG. 6A) and 25° C. (FIG. 6B). An increase in temperature resistance is shown in the crosslinked samples (solid) versus the uncrosslinked samples (dashed). For uncrosslinked samples, mass loss begins around 200° C. and plateaus around 360° C. During the 160° C. change in temperature, the uncrosslinked samples showed an average 39.78±1.18% mass was lost. This is in contrast to the crosslinked samples that were submerged in tCA at 37 C and 25 C, which showed an average 25.07±2.55% and 33.14±2.25% change in mass, respectively, across the same 160° C. change. These changes in mass loss were significantly lower in comparison to uncrosslinked samples, confirming the stabilization of the scaffolds using these crosslinking methods. FIG. 7 shows a graph that can demonstrate the results of a thermogravimetric analysis of a gelatin scaffold crosslinked for 1 day in 10% (v/v) tCA submerged at 37° C. The samples changed an average of 29.90±1.34% in mass across the 160° C. temperature range. This significant reduction in mass loss also confirms increased thermal stability of the scaffolds using this crosslinking method. Scaffolds submerged in tCa solutions at 37° C. experienced less mass loss, therefore more thermal stability in comparison to the sample crosslinked at 25° C. The increased temperature could allow for increased permeation of the solution through the scaffold, resulting in a stronger material. FIGS. 8A-8B show a graph that can demonstrate a summary of the results of the thermogravimetric analyses shown in FIGS. 6 and 7. FIGS. 9A-9B shows a graph that can demonstrate the results of a thermogravimetric analysis of gelatin scaffolds crosslinked in the vapor phase of a 25% tCa solution at 37° C. and 25° C. These samples were unable to be analyzed due to the variability of the mass measurement throughout the experiment. The initial masses of the scaffolds were too low the equipment was unable to accurately detect the change in mass.

### References for Example 2

[1] A. Bigi, G. Cojazzi, S. Panzavolta, K. Rubini, N. Roveri, Mechanical and thermal properties of gelatin films at different degrees of glutaraldehyde crosslinking, Biomaterials. 22 (2001) 763-768. doi:10.1016/S0142-9612(00)00236-2.
[2] S. Farris, J. Song, Q. Huang, Alternative reaction mechanism for the crosslinking of gelatin with glutaraldehyde, J. Agric. Food Chem. 58 (2010) 998-1003. doi:10.1021/jf9031603.
[3] Y. Lui, X. Liang, R. Zhang, W. Lan, W. Qin, Fabrication of electrospun polyactic acid/cinnamaldehyde/b-cyclodextrin fibers as an antimicrobial wound dressing, Polymers (Basel). 9 (2017) 464.
[4] C. Gomes, R. G. Moreira, E. Castell-Perez, Poly (DL-lactide-co-glycolide) (PLGA) nanoparticels with entrapped trans-cinnamaldehyde and eugenol for antimicrobial delivery applications, J. Food Sci. 76 (2011) 216-224.
[5] V. N. Babu, S. Kannan, Enhanced delivery of baicalein using cinnamaldehyde cross-linked chitosan nanoparticles inducing apoptosis, Int. J. Biol. Macromol. 51 (2012) 1103-1108.
[6] M. P. Balaguer, J. Gomez-Estaca, R. Gavara, P. Hernandez-Munoz, Biochemical Properties of bioplastics made from wheat gliadins cross-linked with cinnamaldehyde, J. Agric. Food Chem. 59 (2011) 13212-13220.
[7] H. Gao, H. Yang, Characteristics of poly(vinyl alcohol) films crosslinked by cinnamaldehyde with improved transparency and water resistance, J. Appl. Polym. Sci. (2017) 45324.
[8] Q. Zou, J. Li, Y. Li, Preparation and characterization of vanillin-crosslinked chitosan therapeutic bioactive microcarriers, Int. J. Biol. Macromol. 79 (2015) 736-747.
[9] M.-S. Lee, S.-H. Lee, Y.-H. Ma, S.-K. Park, D.-H. Bae, S.-D. Ha, K.-B. Song, Effect of Plasticizer and CrossLinking Agent on the Physical Properties of Protein Films, Prev. Nutr. Food Sci. 10 (2005) 88-91. doi:10.3746/jfn.2005.10.1.088.
[10] A. H. Dewi, I.D. Ana, J. Jansen, Preparation of a calcium carbonate-based bone substitute with cinnamaldehyde crosslinking agent with potential anti-inflammatory properties, J. Biomed. Mater. Res.—Part A. 105 (2017) 1055-1062. doi:10.1002/jbm.a.35990.
[11] K. Lirdprapamongkol, H. Sakurai, N. Kawasaki, M.-K. Choo, Y. Saitoh, Y. Aozuka, P. Singhirunnusorn, S. Ruchirawat, J. Svasti, I. Saiki, Vanillin suppresses in vitro invasion and in vivo metastasis of mouse breast cancer cells, Eur. J. Pharm. Sci. 25 (2005) 57-65. doi:10.1016/j.ejps.2005.01.015.
[12] J. A. Hoskins, The occurrence, metabolism and toxicity of cinnamic acid and related compounds, J. Appl. Toxicol. 4 (1984) 283-292. doi:10.1002/jat.2550040602.
[13] G. Huang, P. Masi, D. Pandya, S. Amara, G. Collins, T. Arinzeh, An investigation of common crosslinking agents on the stability of electrospun collagen scaffolds, J. Biomed. Mater. Res. A. 103 (2015) 762-771. doi:10.1002/jbm.a.35222.

### Example 3

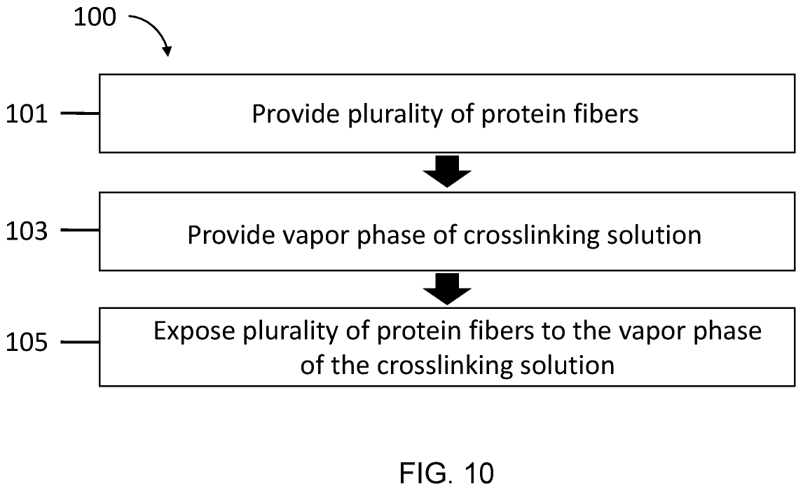

FIG. 10 is a flow chart of a method according to the present disclosure. According to the method 100, a plurality of protein fibers are provided 101. A vapor phase of a crosslinking solution is then provided 103, and the plurality of protein fibers are exposed to the vapor phase of the crosslinking solution 105.

### Example 4

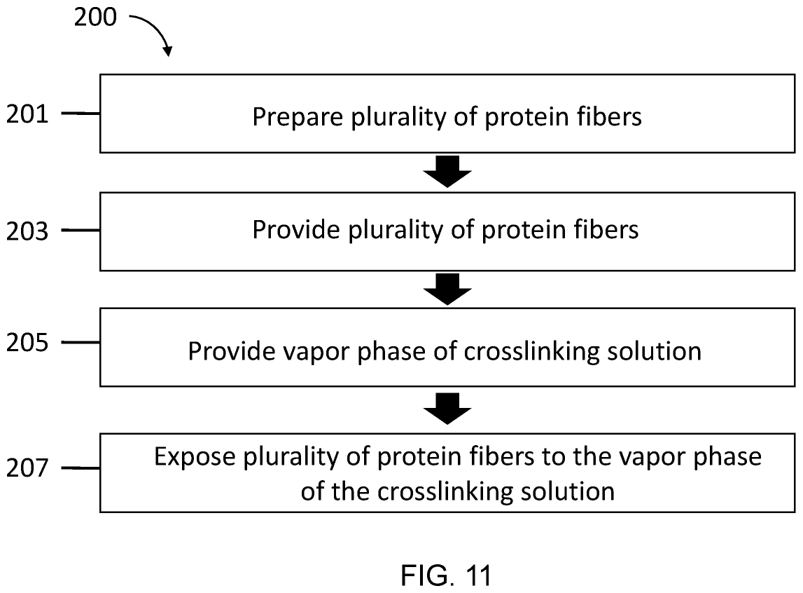

FIG. 11 is a flow chart of a method according to the present disclosure. According to the method 200, a plurality of protein fibers are prepared 201 and then the plurality of protein fibers are provided 203. A vapor phase of a crosslinking solution is then provided 205, and the plurality of protein fibers are exposed to the vapor phase of the crosslinking solution 207.

Ratios, concentrations, amounts, and other numerical data may be expressed in a range format. It is to be understood that such a range format is used for convenience and brevity, and should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figure of the numerical value. In addition, the phrase “about ‘x’ to ‘y’” includes “about ‘x’ to about ‘y’”.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

All publications and patents cited in this specification are incorporated by reference as if each individual publication or patent were specifically and individually indicated to be incorporated by reference and are incorporated by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could differ from the actual publication dates that may need to be independently confirmed.

We claim:

1. A method of crosslinking protein fibers comprising:
exposing a plurality of protein fibers to a vapor phase of a crosslinking solution comprising an amount of a natural aldehyde for an amount of time.

2. The method of claim 1, wherein the amount of the natural aldehyde ranges from about 10% to 25% (v/v) or (w/v).

3. The method of any one of claims 1-2, wherein the natural aldehyde is cinnamaldehyde or vanillin.

4. The method of any one of claims 1-3, wherein the crosslinking solution further comprises ethanol.

5. The method of claim 4, wherein the amount of ethanol ranges from about 75% to about 90% (v/v) or (w/v).

6. The method of any one of claims 1-5, wherein the amount of time can range from about 1 hour to 24 hours.

7. The method of any one of claims 1-5, wherein the amount of time can range from about 1 day to about 14 days.

8. The method of any one of claims 1-7, wherein the protein fibers are collagen and elastin.

9. The method of any one of claims 1-7, wherein the protein fibers are gelatin.

10. The method of any one of claims 1-7, wherein the protein fibers comprise gelatin, elastin, collagen, silk, keratin, soy, or zein, individually or in combination.

11. The method of any one of claims 1-10, wherein the exposing is at a temperature of about 25° C. to about 45° C.

12. The method of any one of claims 1-11, wherein the exposing is at a pressure of about 7.5 PSI to about 30 PSI.

13. The method of any one of claims 1-12, wherein the protein fibers are prepared by electrospinning, wet/dry jet spinning, dry spinning, centrifugal spinning, solution blowing, self-assembly, phase separation, or 3D printing before exposing.

14. A protein scaffold comprising:
protein fibers crosslinked to each other via a natural aldehyde.

15. The protein scaffold of claim 14, wherein the natural aldehyde is cinnamaldehyde or vanillin.

16. The protein scaffold of any one of claim 14 or 15, wherein the protein fibers are collagen and elastin.

17. The protein scaffold of any one of claims 14-16, wherein the protein scaffold is made via the method as in any one of claims 1-8.

18. The protein scaffold of any one of claims 14-17, further comprising a population of cells.

19. The protein scaffold of any one of claims 14-18, further comprising an active agent.

20. A method comprising:
implanting a protein scaffold as in any one of claims 14-19 into a subject.

21. The method of claim 20, wherein the subject has cardiovascular disease.
